(19) United States
(12) Patent Application Publication
(10) Pub. No.: US 2003/0082528 A1
(43) Pub. Date: May 1, 2003

(54) Method to detect the presence of a microorganism or agent in an animal

(75) Inventors: David R. Smith, Davey, NE (US); Laura L. Hungerford, Silver Spring, MD (US); Rodney A. Moxley, Lincoln, NE (US); Susanne Hinkley, Martell, NE (US); Terry Klopfenstein, Lincoln, NE (US); Jeff Gray, Athens, GA (US)

Correspondence Address: SENNIGER POWERS LEAVITT AND ROEDEL, ONE METROPOLITAN SQUARE, 16TH FLOOR, ST LOUIS, MO, 63102, US

(73) Assignee: The Board of Regents for the University of Nebraska

(21) Appl. No.: 10/213,998

(22) Filed: Aug. 7, 2002

Related U.S. Application Data
(60) Provisional application No. 60/310,706, filed on Aug. 7, 2001.

(51) Int. Cl. C12Q 1/70; C12Q 1/04

(52) U.S. Cl. 435/5; 435/34

(57) ABSTRACT

The present invention provides a method to detect the presence of a microorganism or agent in an animal. The method encompasses placement of devices at various locations where the animal resides so as to induce the animal to initiate contact with the device. As a result of this contact, the animal deposits various microorganisms and agents on the device. The device is then tested for the presence of the particular microorganism or agent of interest.

### CROSS REFERENCE TO RELATED APPLICATION

This application claims priority from Provisional Application Serial No. 60/310,706 filed on Aug. 7, 2001, which is hereby incorporated by reference in its entirety.

### FIELD OF THE INVENTION

The present invention generally relates to a method for detecting the presence of a microorganism or agent in an animal. More particularly, the method provides a means to detect the presence of a microorganism or agent in a group of animals by isolating a target microorganism or an agent from a device.

### BACKGROUND OF THE INVENTION

Food-borne diseases are an important public health concern. In the United States alone, the occurrence of food-borne illness is estimated to be between 6 to 80 million illnesses with approximately 9000 deaths annually. The most prevalent agent causing food-borne illness is Campylobacter jejuni. This agent alone is estimated to be responsible for causing 4 million of these cases with more than 1000 deaths annually. Although less prevalent than Campylobacter jejuni, Salmonella (non-typhoid) is also a major health concern because it is responsible for 2 million cases of disease and approximately 2000 deaths each year. Further, more than 75,000 of these cases with more than 61 deaths annually have been attributed to food-borne Escherichia coli 0157:H7. And while the incidence of disease due to E. coli 0157:H7 is much more rare than many other food-borne pathogens, such as Salmonella and Campylobacter, it is a particular concern because it is often life-threatening in children and the elderly.

In addition to the staggering health concerns associated with food-borne illness, is the severe economic burden these agents cause. Moreover, this economic burden is not limited to one specific area of the economy, but stretches through several sectors. For example, human health costs associated with food-borne illness is estimated to be approximately 22 billion dollars annually in the United States alone. Equally devastating are the somewhat intangible costs incurred by the livestock industry, such as negative public perception that may cause a reduced demand for beef.

Animals have been identified as a major source of these food-borne illnesses when humans consume meat and other products contaminated with microorganisms at slaughter. Food processors and government agencies have responded to this problem by instituting hazard analysis/critical control point (HACCP) models to provide food safety assurance. HACCP systems are designed to systematically prevent food safety hazards from occurring. And while HACCP has resulted in a decreased risk of contamination during the post-harvest period, it does not address the issue of contamination caused in the pre-harvest period.

There is growing consensus that microorganism control based on risk assessment from the farm to table is the most effective strategy for reducing human food-borne illness. Information on scientifically based control strategies for the ranch/farm level, however, is currently lacking. This lack of information is in part attributable to the inability to successfully and efficiently monitor individual animals. One reason for this is the difficulty of determining the infection status of animals at any point in time. Problems in diagnosing result from the fact that the presence of some microorganisms, such as E. coli 0157:H7, in animals occurs in most cases without the manifestation of clinical signs. Equally, these problems exist because there is a lack of reliable methods to monitor animals for food-borne microorganisms.

One method currently employed to detect the presence of microorganisms in animals is to test individual animals by culturing fecal samples collected from each animal. Sufficient sampling of individual animals to make inferences regarding a group of animals, however, is enormous and is impractical in commercial settings because of the time, expense, labor and potential detriment to the animals. Even if time, expense, labor or culture methodology were not issues, the microorganism status of groups of animals might be incorrectly classified by culturing only some animals within the group.

Therefore, for effective field studies to determine risk and control factors, it must be determined whether microorganisms are present and/or prevalent in a group of animals, either because the animals in the group are colonized or the environment in which they are housed is contaminated. The group level is the unit of interest in most animal production systems because this is the level that monitoring and managing health is practical. Therefore, most conceivable control points for reducing human food-borne microorganisms in feedlot animals would be directed toward groups of animals rather than individuals.

Accordingly a need exists for a reliable, cost efficient test that can be conducted at the ranch/farm level in order to monitor the microorganism status of groups of animals prior to marketing to evaluate control points and take corrective actions if necessary. The present invention addresses this need by providing a method to detect the presence of a microorganism in a group of animals that is both reliable and cost efficient.

### SUMMARY OF THE INVENTION

Among the several aspects of the invention, therefore, is provided a method to detect the presence of a microorganism or agent in an animal. The method comprises placing a device at a location within an area in which the animal resides to induce the animal to initiate contact with the device and then determining the presence of the microorganism or agent on the device.

In one aspect of the invention, the method is employed to detect the presence of a microorganism that is a human food-borne pathogen.

In still a further aspect of the invention, the method is employed to monitor the health status of an animal or a group of animals.

### BRIEF DESCRIPTION OF THE DRAWINGS

These and other features, aspects, and advantages of the present invention will become better understood with regard to the following description, appended claims and accompanying figures where:

FIG. 1 depicts the percent of cattle making contact with either 3 or 7 ropes placed per pen within 2 hours of observation.

FIG. 2 depicts the ability of E. coli 0157:H7 and S. typhimurium to survive on the sampling devices indicated.

FIG. 3 depicts the ability of E. coli 0157:H7 and Salmonella to survive on the sampling devices indicated.

FIG. 4 depicts the relationship of feedlot pen prevalence of fecal shedding of E. coli 0157:H7. Each different bar shade represents a different feedlot.

FIG. 5 depicts the relationship of pen-test rope and composite fecal culture results to the percent of cattle in the pen shedding E. coli 0157:H7.

FIG. 6A is a graphic representation of E. coli 0157:H7 rope-positives per pen-week of Study 1.

FIG. 6B is a graphic representation of E. coli 0157:H7 rope-positives per pen-week of Study 2.

FIG. 7A is a graphic representation of Salmonella spp. rope-positives per pen-week of Study 1.

FIG. 7B is a graphic representation of Salmonella spp. rope-positives per pen-week of Study 2.

FIG. 8 depicts a cow contacting a rope device employed in the method of the invention.

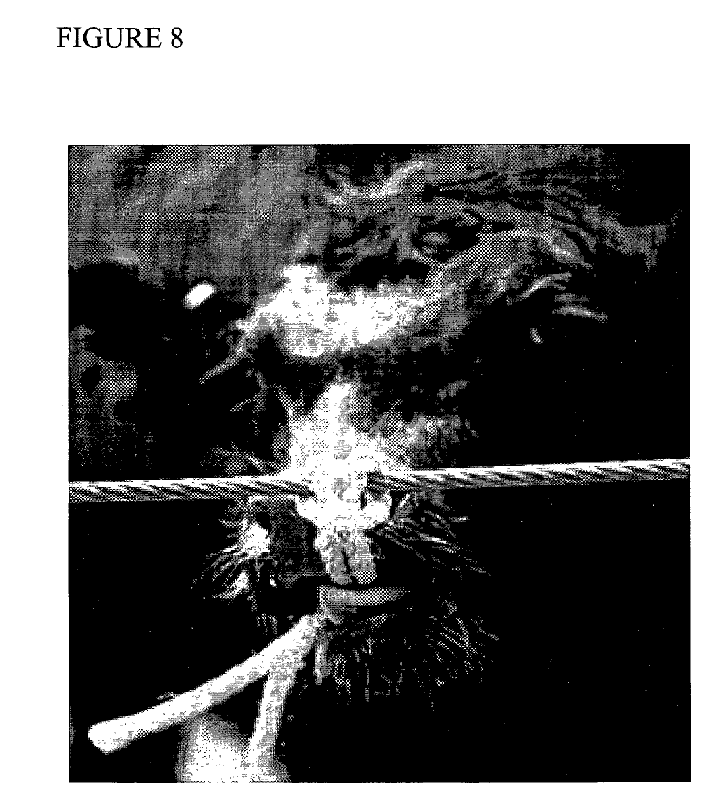

### ABBREVIATIONS AND DEFINITIONS

To facilitate understanding of the invention, a number of terms and abbreviations as used herein are defined below:

“Prevalence” shall mean the percentage of animals in a group at a given point in time that are infected with the microorganism of interest.

“Food-borne Pathogen” shall include any microorganism that is pathogenic to humans when present in food that is consumed.

“Sampling Device” and “Device” are used interchangeably herein and mean one or more devices as described in further detail herein.

Areas where the animals are “housed” or “reside” means any area where the animal or group of animals are located. For farm animals, such as cattle, this may include without limitation a feedlot, pen or pasture. For wild animals not in captivity, however, this may include the range or territory where the animal or group of animals are located.

### DESCRIPTION OF THE PREFERRED EMBODIMENTS

Applicants have discovered an efficient, effective method to determine the microorganism status of a group of animals. This method, unlike previous approaches, focuses on detection at the group level rather than at the individual level. Detection at the group level is advantageous because in most livestock production systems it is at this organizational level at which monitoring and managing animal health is practical. Moreover, the current method is also highly beneficial because it does not require human-handling of individual animals prior to shipping which often results in economic loss due to shrink, dark cutters, and bruising.

One aspect of the present invention provides a method to detect the presence and/or prevalence of a microorganism in a group of animals. Yet another aspect of the invention provides a method to detect the presence of an agent in a group of animals. Irrespective of the particular embodiment, the method encompasses placement of devices at various locations where the animals reside so that one or several animals in the group are induced to initiate contact with the device. As a result of this contact, the animal deposits a sample on the device that contains various microorganisms and agents. The device is then tested for the presence of the particular microorganism or agent of interest. A positive test indicates that one or more animals in the group are colonized or hide-contaminated with the microorganism or agent.

A basic premise relied upon in the present invention is that animals are naturally curious. So, when confronted with a new object, they approach, sniff, lick and eventually chew the object or rub against the object with their body. With this rudimentary knowledge of animal behavior, Applicants have designed various sampling devices for use in the current method that preferably heighten the animal's curiosity for the device and thus, induce the animal into making contact with the device. Applicants have discovered that the degree of animal curiosity for the device is influenced by several features, including, but not limited to the device's size, shape, color, and type of material. Of course, different animal species are attracted to devices with varying combinations of these features. One skilled in the art can readily design sampling devices that maximize each particular animal species' curiosity for the device based upon behavioral characteristics indigenous to the particular species.

In addition to inducing the animal to make contact, the device is preferably durable. This durability is necessary because the device is contacted routinely by one to several animals in the group. The device, accordingly, should be able to withstand both the frequency and force of this repeated contact. The degree of durability required will be highly dependent upon the particular animal species. Generally speaking, however, larger and more active animal species, such as cattle, require a device that is more durable than smaller and less active species, such as sheep.

In embodiments where the presence of a microorganism is detected, the device employed should also facilitate survival of the particular microorganism. As detailed above, the microorganism is deposited on the sampling device when the animal contacts the device either by making nasal and oral contact with the device or by rubbing against the device with a part of its body. After being deposited on the sampling device, the microorganism preferably is able to attach to and survive on the device for a few hours to several days, weeks or even months depending on the particular embodiment. Applicants have found that devices constructed from porous materials such as fabric, cloth, plastic or various other polymers typically enhance survival of the microorganism.

Generally speaking, any device that induces the animal to make contact, and is durable may be employed. Additionally, in embodiments where the presence of a microorganism is detected, preferably the device selected is one which facilitates survival of the microorganism to be detected. By way of example, in one embodiment the device is a rope made of cotton, braided nylon, or natural fiber. In yet another embodiment, the sampling device is a ball. In still another embodiment, the sampling device is a sponge. And in another embodiment, the device is artificial turf. Of course, the particular device employed may vary considerably from these specific devices without departing from the scope of the present invention. Moreover, when more than one device is utilized, any combination of different types of devices may be employed.

For embodiments where the method will be employed to monitor animals over an extended period of time, the type of device used is may be changed on a frequent basis. By changing the type of device every several days or weeks, animals' curiosity towards the device is maintained, thus continually inducing the animals' contact with the device. For example, if the initial device used is a ball, the ball may be used for a week and then the next week a rope may be employed and the week after that, a piece of artificial turf may be utilized. One of ordinary skill in the art can readily determine a desirable frequency of changing a device based upon the observation of animals in the presence of the device.

The sample may be collected from the animal with the device by any means generally known in the art. In one embodiment, the device is used to directly collect a sample from the animal when the animal makes contact with the device by chewing, rubbing or licking. For example, the device may be a rope that the animal chews, rubs or licks. After the animal contacts the rope, the rope is collected and tested for the microorganism or agent of interest as detailed herein. In yet another embodiment, the device is used to collect and transfer a sample from the animal. By way of example, in this embodiment the device may be a ball. The animal makes contact with the ball by rubbing, licking or chewing the ball. The ball may then be contacted with a second device or a transport media. In this embodiment, the transport media or second device is then collected and analyzed for the presence of the microorganism or agent of interest as detailed herein. One skilled in the art can readily select a suitable means to collect a sample from the animal.

After the particular type of device and collection method are selected based upon the criteria above, one or more devices are placed in the area the animals reside. In general, about one to about twenty sampling devices are placed in the area the animals are housed. More preferably, however, about one to about ten sampling devices are utilized and still more preferably, about 3 to about ten devices are employed. The device is also preferably placed in an area that the animals frequently and routinely visit in order to maximize the likelihood or to entice one to several animals to make contact with the device. One skilled in the art, again employing basic principles of animal behavior, can readily select these high traffic areas. These areas, however, typically include areas where the animal's feed, water, or shelter are located. For example, the sampling device may be attached to a feedbunk or water tank. The means of attachment is generally not a critical feature of the invention to the extent that the sampling device cannot be easily removed by the animals.

Because animals are generally more active at certain times of the day, another aspect that impacts the likelihood of one or several animals making contact with the device, in addition to its location, is both the time and length of time that the device is placed in the area the animals reside. Moreover, in embodiments involving the detection of a microorganism, both the time and length of time the device is placed is an important aspect because it may impact the ability of microorganisms to survive on the device. Typically, therefore, the device will be placed during the time of day and for a length of time that corresponds to the period of greatest animal activity. But yet also during the time of day and for a length of time so that microorganisms are able to survive on the device. By way of illustration, Applicants have found that cattle are most active in the 2 hours prior to sunset (“night-time period”) and E. coli 0157:H7 are able to survive on the rope devices during this time and overnight (as described in the examples below). In some embodiments, however, depending upon the agent or microorganism to be detected, the device may be collected several weeks or even months after its initial placement. More preferably, the device is collected between 2 to about 8 hours after initial placement. Of course, the optimal time for placing and collecting the device may vary considerably from one animal species to another and one skilled in the art can select this time.

In order to minimize potential contamination and decrease experimental error, the devices are preferably aseptically placed, collected, stored and tested. Aseptic conditions may be maintained employing principles of sterile technique generally known to those skilled in the art.

After its collection, the sampling device may then be tested for the presence of the particular microorganism or agent of interest. As detailed above, however, a number of microorganism in addition to the microorganisms of interest may be present on the sampling device. Accordingly in a preferred embodiment, the method employed for detection typically will involve a series of culture techniques designed to inhibit growth of undesirable microorganisms, while enhancing the growth of the target microorganism. These culture techniques generally involve the addition of a compound to the culture media, such as an antibiotic or a particular nutrient, that results in selection of the target microorganism. For example, Applicants have found that the addition of brilliant green bile broth to the culture media, as described in more detail below, positively selects for Escherichia coli 0157:H7 and Salmonella, two target microorganisms of particular interest. Moreover, any other means generally known may be employed to further select for the microorganism of interest, such as an agglutination test. In addition, the polymerase chain reaction and/or DNA sequencing may be utilized to further characterize and confirm the identity of the target microorganism.

The method of the present invention may be employed to detect the presence of a microorganism or agent in any animal. Typically, however, the animal species is cattle, sheep, swine, goats, horses, bison, deer, companion animals such as dogs or cats, any animal typically housed in a zoo, or any animal generally classified as a wild animal. In a preferred embodiment, the animal species is cattle.

One aspect of the invention provides a means to detect the presence of any microorganism that is deposited on the device by the animal. Typically, the microorganism is a bacteria, virus, protozoa or fungi. In a preferred embodiment, the microorganism is a bacterium. In an even more preferred embodiment, the microorganism is a food-borne human pathogen such as Campylobacter jejuni, Salmonella, Escherichia coli, Listeria monocytogenes, and Vibrio. Still in a more preferred embodiment, the food-borne microorganism is Escherichia coli 0157:H7 or Salmonella.

In another aspect of the invention, the method may be employed to detect the presence of a microorganism that is pathogenic to the animal species. In this embodiment, the method may be used as a tool to monitor animal health status. In embodiments where the animal is a cow, examples of microorganisms that affect animal health include but are not restricted to foot and mouth disease virus, bovine diarrhea virus, pseudorabies virus, and Salmonella spp. In embodiments where the animal is a pig, examples of microorganisms that affect animal health include but are not restricted to pseudorabies virus, swine influenza virus, and Mycoplasma hyopneumoniae. In embodiments where the animal is a horse, examples of microorganisms that affect animal health include but are not restricted to Streptococcus equi, equine herpesvirus 1, and Pseudomonas spp. Moreover, in embodiments where the animal is a cat, examples of microorganisms that affect animal health include but are not restricted to feline enteric coronavirus, feline calicivirus, and feline leukemia virus.

A further aspect of the invention provides a means to detect the presence of an agent present in the animal's environment. Any agent may be detected to the extent it is present in detectable quantities in the animal's oral cavity or on the animal's hide. In one embodiment the agent is a chemical substance. The chemical substance comprises, without limitation, arsenic, organophosphate, organochlorine, carbamate, antibiotic, or solvent. It should be noted that these agents may not be harmful to the animals that come in contact therewith, but could present a danger for humans if they are present in food products derived from such animals. Accordingly, in addition to monitoring the health of animals, the detection of these agents is important for preventing contamination of food products that are used for human consumption.

In another embodiment, the agent is a mycotoxin, such as, e.g., aflatoxin, vomitoxin, or zearalone. Mycotoxins are toxic substances produced by fungi (molds) that can grow on grain, feed, or food. A number of these substances exhibit considerable pathology in both animals and humans. For instance, aflatoxin is a liver toxin and a carcinogen. In swine, aflatoxin can cause reduced weight gain, hepatitis, and death. Consumption of aflatoxin can result in acute aflatoxicosis in humans, which is characterized by vomiting, abdominal pain, pulmonary edema, convulsions, and death in some cases. Zearalenone is an estrogenic mycotoxin and can affect reproduction. In swine, feed contaminated with zearalenone can cause infertility, abortion and other breeding problems. The presence of vomitoxin in animal feed can also cause reduced animal feeding and weight gain. Thus, the ability to readily detect the presence of mycotoxins in animals plays a role in both animal and human health.

Agents, as described herein, can be detected by a number of assays that are known in the art. These assays include, but are not limited to mass spectrometry, high pressure liquid chromatography (HPLC) and gas chromatography (GC). By way of example, the detection of the organophosphate insecticide chlorpyrifos can be performed by utilizing gas chromatography to analyze the contents of the rope or any other device of the present invention that contains at least one animal's salivation thereon.

### EXAMPLES

In the examples described below, unless indicated otherwise, the following sampling procedures were used.

Sample Collection Techniques

Rope sampling—75 cm lengths of 1.3 cm diameter manilla rope were folded double and fastened over feedbunks and water tanks using plastic cable ties. Ropes were applied and removed using aseptic techniques (sterile gloves) and transported to the lab in double wrapped sterile containers (Whirl-pak® bags over inverted plastic sleeves).

Water tank sampling—A gloved hand and arm was used to scrape 3 sterile 100 ml containers along the bottom of the water tank to collect water and sediment. The containers were sealed and then double wrapped in the inverted sleeve.

Fecal pat sampling—From each pen, approximately 5 g each from 20 fresh fecal pats were collected using a sterile glove and placed as a composite sample into a sterile plastic container.

Feed sampling—Approximately 1 liter of feed was collected from each feedbunk by grabbing 50 ml sub-samples intermittently from the length of the bunk with sterile gloves and transported to the lab within 24 hours in a double wrapped plastic container.

Individual animal feces sampling—Approximately 30 g of feces were collected from the rectum of cattle restrained in a handling chute during routine management procedures (re-implanting). The fecal sample was placed in a sterile collection cup.

Oral swab sampling—Swab samples were collected from the oral cavity of cattle restrained in a handling chute during routine management procedures using methods generally known to those of ordinary skill in the art.

### Example 1

### Animal Behavior Study

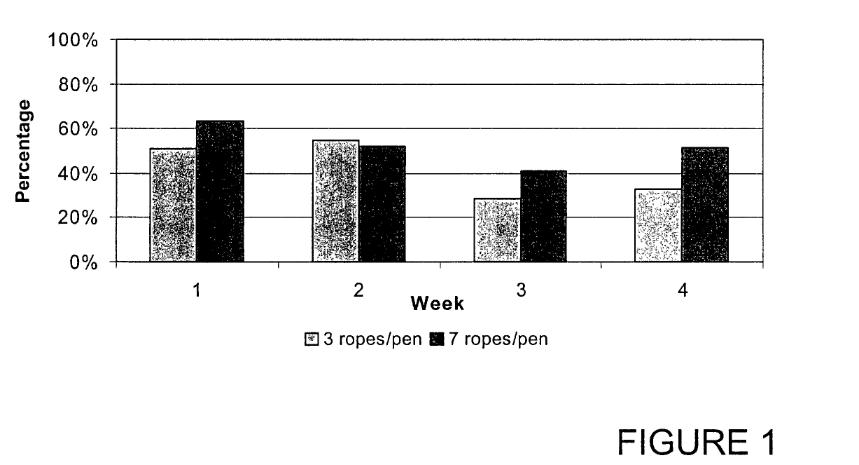

Rope sampling devices were aseptically placed in cattle feedlot pens by fastening them with plastic cable ties over feedbunks and water tanks. Either 3 devices/pen or 7 devices/pen were placed in 8 pens in the evening and aseptically collected the following morning to match the period of greatest cattle activity and to avoid daytime heat and sunlight. Cattle activity was observed and recorded during this period. In the late evening hours it was not unusual for cattle to make contact with the device within 5 minutes of placing the device in the pen. The percentage of cattle to make contact with the devices within a 2-hour period of observation in 8 pens of cattle is summarized in FIG. 1.

### Example 2

### Evaluation of the Adequacy of a General Culture Method in Detecting E. coli 0157:H7 and Salmonella in Various Feedlot Samples

Swabs were obtained from the mouths of 20 feedlot cattle. In addition, ropes that had been placed in the pen overnight and fecal pat samples from pen floors were collected. All samples were cultured in gram negative (GN) broth with vancomycin, cefixime and cefsulodin added to the enrichment broth. Samples were incubated at 37° C. for 6 hours and subsequently, 1 ml of the enrichment was subjected to immunomagnetic separation.

Immunomagnetic separation was accomplished using Dynal Immunomagnetic Isolation kit following the manufacturer's directions. Briefly, 20 μl of Dynabeads anti-E. coli 0157:H7 was added to the enrichment. Next, after incubating for 30 minutes at 26° C. with constant gentle rotation on a rotating rack, the beads were separated using a magnetic field and washed twice with phosphate buffered saline with Tween. Fifty μl of the recovered beads were plated onto sorbitol-MacConkey agar with cefixime and tellurite added (CT-SMAC). After an 18-hour incubation at 37° C., the plates were overgrown with environmental bacterial flora. The flora was characterized using techniques generally known to those of ordinary skill in the art. Stocks of the cultures were made for further testing. Thirteen different bacterial species were recovered from the 0157:H7 immunomagnetic separation and enrichment protocol. These species were: Enterobacter sakazakii, Enterobacter agglomerans, Enterobacter cloacae, Enterobacter taylorae, Klebsiella oxytoca, Klebsiella pneumoniae, Escherichia coli, Escherichia hermannii, Kluyvera cryocrescens, Pseudomonas putida, Proteus vulgaris, Shewanella putrefaciens and Bacillus licheniformis. The predominant flora found in these samples included Escherichia hermanni, Enterobacter cloacae, Enterobacter amnigenus, Proteus vulgarus, Bordetella spp., and Pseudomonas aeruginosa.

### Example 3

### Confirmation of the Effectiveness of a Method Specifically Adapted to Inhibit Undesirable Flora from Certain Samples while Retaining Favorable Culture Conditions for E. coli 0157:H7 and Non-typhoid Salmonella

The flora encountered in feedlot environmental samples and cattle mouths is considerably different than that found in feces and therefore many of these bacteria are positively selected by even the most sensitive and selective culture methods. This experiment tested a method to exclude this flora while positively enriching the samples for the target E. coli 0157:H7 and Salmonella organisms.

Rope samples and mouth swabs were taken using the procedures outlined above. The ropes and mouth swabs were added to a brilliant green bile (BGB) broth containing 2% bile and 0.0013% brilliant green while approximately maintaining a 1 g of sample to 10 ml of media ratio. The samples were placed in an incubator at 37° C.

E.coli 0157:H7 Isolation and Confirmation Procedure:

After 6 hours of incubation at 37° C., 1 ml of sample was removed and placed into the Dynal anti-0157:H7 immunomagnetic separation protocol discussed in example 2. Fifty μl of the final resuspension was plated on sorbitol-MacConkey agar with cefixime and tellurite added (CT-SMAC) and incubated 18 hours at 37° C. Sorbitol-negative suspect colonies were picked and transferred to a 96-well MUG/MAC plate using a sterile toothpick. The MUG/MAC plate was read and suspect isolates were transferred to blood plates which were then incubated for 24 hours at 37° C. Next, each isolate was tested using the Remel RIM E.coli 0157:H7 agglutination test following the manufacturer's directions. Cultures of each isolate that tested positive for 0157:H7 antigen were transferred to 100 μl of distilled water and boiled in a heat block at 100° C. for 5 minutes. This lysate was then used to reconfirm 0157:H7 agglutination test results and was further tested by PCR. PCR confirmation of each isolate was determined by amplification of specific genetic markers (i.e. wbdn, stx, and eae genes).

Salmonella spp. Isolation and Confirmation Procedure:

After removing 1 ml of the sample for the E. coli isolation and confirmation, the sample was returned to the incubator. After a total incubation period of 24 hours at 37° C., 1 ml of the BGB enrichment culture was transferred to a tube containing tetrathionate broth and processed as previously described in Fedorka-Cray et al., 1998 (Survey of Salmonella serotypes in feedlot cattle, Journal of Food Protection 61:525-530). This process involved incubating the TET tube at 37° C. for 48 hours, then inoculating a tube containing Rappaport Vassiliadis R-10 broth (RAP) from the TET broth using a sterile swab. The RAP tubes were then incubated at 37° C. for 24 hours. Next, a sterile inoculating loop was used to inoculate half of an XLT4 plate and streak the other half of the plate. After the XLT4 plates were incubated at 37° C. for 24 hours, any suspect colonies were picked using a sterile wire pick and transferred to a Triple Sugar Iron Agar (TSI) slant tube and a Lysine Iron Agar (LIA) slant tube. These tubes were then incubated at 37° C. for 24 hours. Any positives were tested using the Difco Salmonella Poly O agglutination test following the manufacturer's directions.

### Example 4

### Comparison of Using Oral Versus Rectal Sampling to Classify the E. coli 0157:H7 Status of Individuals and Pens of Feedlot Cattle

Rectal fecal samples and cotton gauze swabs of the oral cavity were collected using the procedures described above for 196 feedlot cattle in 22 pens containing 8-10 animals each. The oral-swab samples were cultured using the method described in example 3. Approximately 10 g of the fecal material was transferred from the collection cup to a Whirl-pak® bag and 100 ml of Gram Negative Hajna broth with vancomycin, cefixime and cefsulodin (GN V/C/C) was added. The samples were incubated at 37° C. for 6 hours. Following incubation, the E. coli 0157:H7 identification procedures described in example 3 were performed.

E. coli 0157:H7 was isolated from both feces and oral swabs of 26 animals, from only the feces of 41 animals, and only the oral swabs from 28 animals. The organism was not isolated from either sample from 101 animals. The agreement between rectal and oral sample results was only slightly beyond that expected by chance (Kappa=0.179, p<0.05). E. coli 00157:H7 was isolated from feces from at least 1 animal in 16 pens, and from oral swabs from at least 1 animal in 19 pens.

### Example 5

### Evaluation of the Ability of Various Sampling Devices to Retain and Promote the Recovery of Certain Microorganisms

This experiment tested the ability of E. coli 0157:H7 and Salmonella to survive on various sampling devices. The three devices chosen were: (A) braided nylon rope, (B) natural fiber manilla rope and (C) cotton rope. Each type of sampling device was submerged in suspension of 2×10⁴ cfu/ml of log phase E. coli 0157:H7 strain ATCC 43895. Ropes were quantitatively cultured using gram negative (GN) broth, and sorbitol-MacConkey agar with cefixime and tellurite added (CT-SMAC) plates using the 5 tube mpn method as previously described in Gray et al., 1995 (Influence of inoculation route on the carrier state of Salmonella choleraesuis in swine, Veterinary Microbiology 47:43-59).

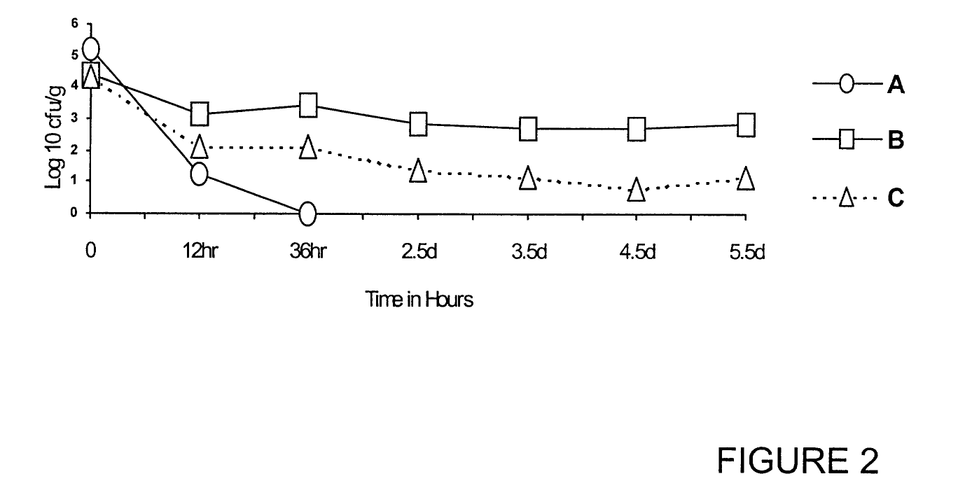

E. coli 0157:H7 was found to survive well on sampling device B, declining only approximately 1.5 logs over a 5.5 day period. We observed larger declines on sampling device C with an approximately 3.5 log decline over 5.5 days. Sampling device A had the highest initial counts of 0157:H7, nearly 1 log higher than the other devices. This is due to the fact that sampling device A retained approximately 115% of the ropes weight in water, whereas sampling devices B and C retained approximately 20%. However, despite the initial high counts, the 0157:H7 numbers declined approximately 4 logs in 12 hours and to undetectable levels within 36 hours. The results of this experiment are shown in FIG. 2.

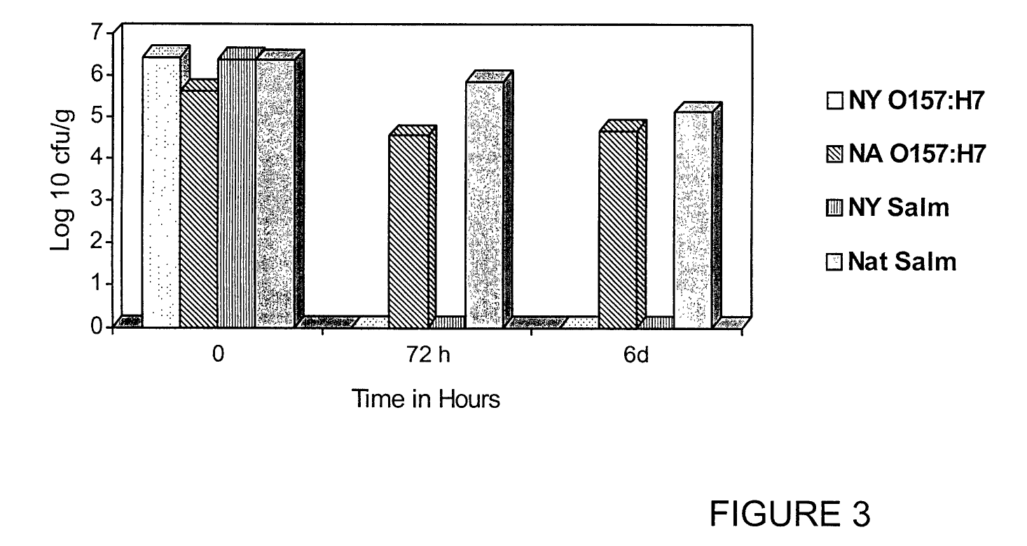

A second experiment was performed utilizing sampling device A and sampling device B with both E. coli 0157:H7 and S. typhimurium to determine if the same results could be observed with Salmonella spp. Briefly, the sampling devices were submerged in phosphate buffered saline (PBS) containing 2×10⁷ cfu/g of the respective organism. The sampling devices were cultured immediately, at 72 hours and at 6 days post inoculation using quantitative measures described above. The results of this experiment are shown in FIG. 3.

### Example 6

### Validation of a Method of Culturing the Sampling Devices to Detect E. coli 0157:H7 from Among the Flora Present in Feedlot Pens

To evaluate the pen test culture methods' ability to detect target organisms from among the natural flora present in feedlot pens, natural fiber manilla ropes (sampling device B from example 5) were placed in feedlot pens containing cattle for approximately 15 hours (overnight). Next, the ropes were inoculated with 1×10² cfu/cm of E. coli 0157:H7 and 3×10² cfu/cm S. typhimurium. The ropes were stored overnight at 26° C. in a biologically secure location. The ropes were subjected to the pen test culture methods as described in example 3. The culture methods successfully recovered the E. coli 0157:H7 and the S. typhimurium inoculum after 18 hours of storage. Uninoculated control pieces of the pen ropes were negative for the target organisms.

### Example 7

### Determining the Prevalence of E. coli in Commercial Feedlots

Twenty-nine commercial feedlot pens from FIVE Midwestern feedlots were Included in the study. Pen size ranged from 36 to 231 (median 107) cattle. Seven ropes (sampling device B in example 5) were placed in each pen the evening prior to sample collection. Feces were collected from the rectum of all cattle in each pen and concurrent rope samples were collected. Additionally, a composite sample of 20 fresh fecal pats from the pen surface, water from water tanks, and partially consumed feed from feedbunks were collected and tested.

Culture methods were specific to the type of sample collected. The culture method for the oral swab and rope samples is provided in example 3. The culture method for the rectal feces sample is described in example 4. The composite fecal sample was mixed well and 10 g of the sample was enriched and analyzed using the same procedure outlined in example 4 for rectal feces samples. The water samples were poured into a filtra-bag and 10 ml of 10×Brilliant green bile (BGB) was added to the sample. Then, the water samples were analyzed using the same procedure used for analyzing the swab and rope samples outlined in example 3. One-third of the feed sample was placed into a filtra-bag and approximately 300 ml of BGB was added. Then, the feed sample was analyzed using the same procedure used for analyzing the swab and rope samples outlined in example 3.

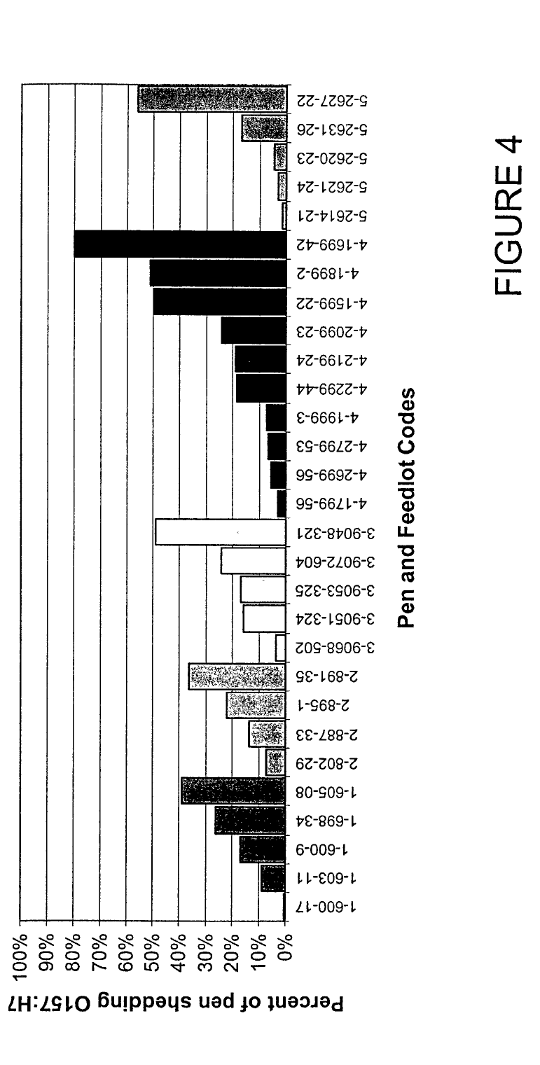

The results of the experiment are shown in FIG. 4. E. coli 0157:H7 was isolated from 714 of 3162 cattle tested (23%) including at least 1 animal from all 29 pens. The pen prevalence of cattle shedding detectable levels of the organism varied widely ranging from 0.7% to 79.8% (median 17.1%). Feedyards did not differ by pen prevalence (Kruskal-Wallis P>0.10); however, the pen prevalence differed widely within feedyards (chi square P<0.001).

### Example 8

### Comparison of the Accuracy of Sampling Device B and Composite Fecal Sampling in Predicting Pen Prevalence of Fecal Shedding and Differentiating High Prevalence Pens from Low Prevalence Pens

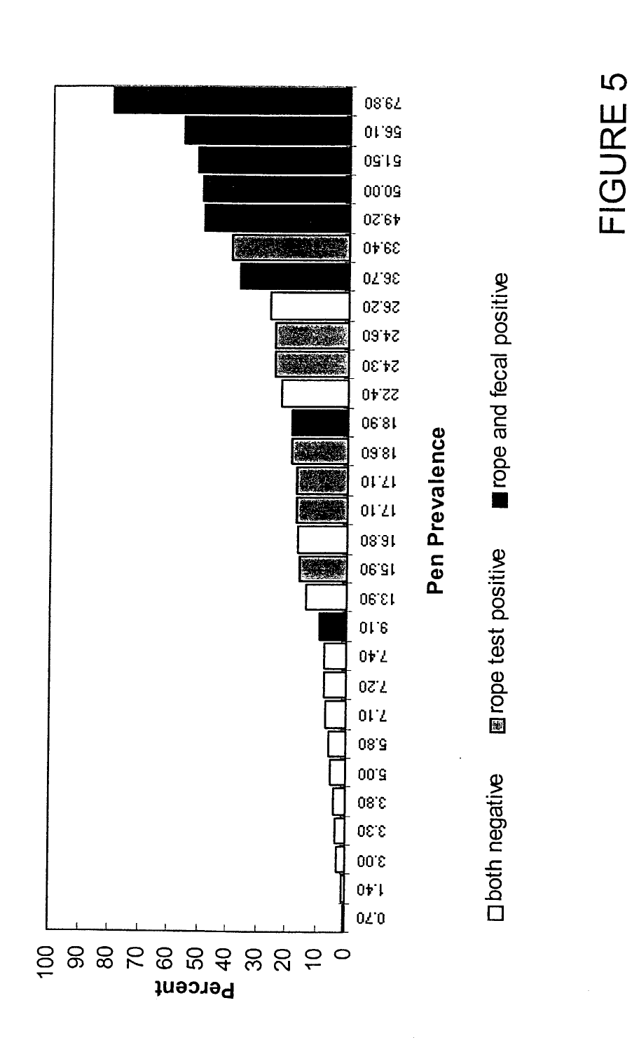

Culture results from rope (sampling device B described in example 5) and composite fecal samples were evaluated as potential pen-tests to predict pen prevalence of fecal shedding. E. coli 0157:H7 was recovered from at least one rope from 15 pens and from the composite fecal sample from 8 pens. Recovery of E. coli 0157:H7 from at least one rope or composite fecal sample was more likely to occur from the higher prevalence pens (Wilcoxon rank sums p=0.001). Ropes and composite feces were evaluated as pen-tests to differentiate high prevalence pens from low prevalence pens. Ropes were optimally efficient (greatest percentage of pens classified correctly) when pens were distinguished as high or low prevalence at a cut-off point of 16% prevalence. Composite feces were optimally efficient when pens were distinguished as high or low prevalence at a cut-off point of 37% prevalence. Only one pen was classified as positive for Salmonella spp based on either the pen-test or composite fecal samples. Salmonella were isolated from individual fecal samples from only 9 of 3162 cattle tested (0.3%) in 5 of the 29 pens (17%). These results are summarized in FIG. 5.

### Example 9

### Feedlot Studies

The presence of food safety pathogens was tested in two separate studies by utilizing rope sampling devices. Study 1 contained five feedyards that included a total of 31 feedlot pens, with a mean pen size of 157 cattle/pen (ranging from 59 cattle/pen to 282 cattle/pen). The mean observation period in Study 1 was 20 weeks (ranging from 15 to 26 weeks). Study 2 also consisted of five feedyards, which included a total of 24 pens, with a mean pen size of 11 cattle/pen (ranging from 46 to 203 cattle/pen). The mean observation period in this study was 18 weeks (from 11 to 25 weeks).

Rope sampling devices were aseptically placed in cattle feedlot pens by fastening them with plastic cable ties over feedbunks and water tanks. 7 devices/pen were placed in pens in the evening, two hours before sunset, and aseptically collected the following morning to match the period of greatest cattle activity and to avoid daytime heat and sunlight. Cattle activity was observed and recorded during this period.

Rope samples were taken using the procedures outlined above. The ropes were added to a brilliant green bile (BGB) broth containing 2% bile and 0.0013% brilliant green while approximately maintaining a 1 g of sample to 10 ml of media ratio. The samples were placed in an incubator at 37° C. E.coli 0157:H7 and Salmonella spp. isolation and confirmation procedures were performed as described in Example 3; however, other methods that are known in the art for isolating food pathogens may also be used.

In Study 1, 627 pen-weeks were observed, wherein 274 (44%) tested positive for E. coli 0157:H7. In Study 2, 425 pen-weeks were observed, wherein 177 (42%) tested positive employing the method of the invention. There was no significant statistical difference between the two studies (p=0.55). The number of pen-weeks, as used herein, was calculated according to the following formulas:

Study 1:

i=31

pen-week=Σpenᵢ×(number of weeks penᵢ was observed)

i=1

Study 2:

i=24

pen-week=Σpenᵢ×(number of weeks penᵢ was observed)

i=1

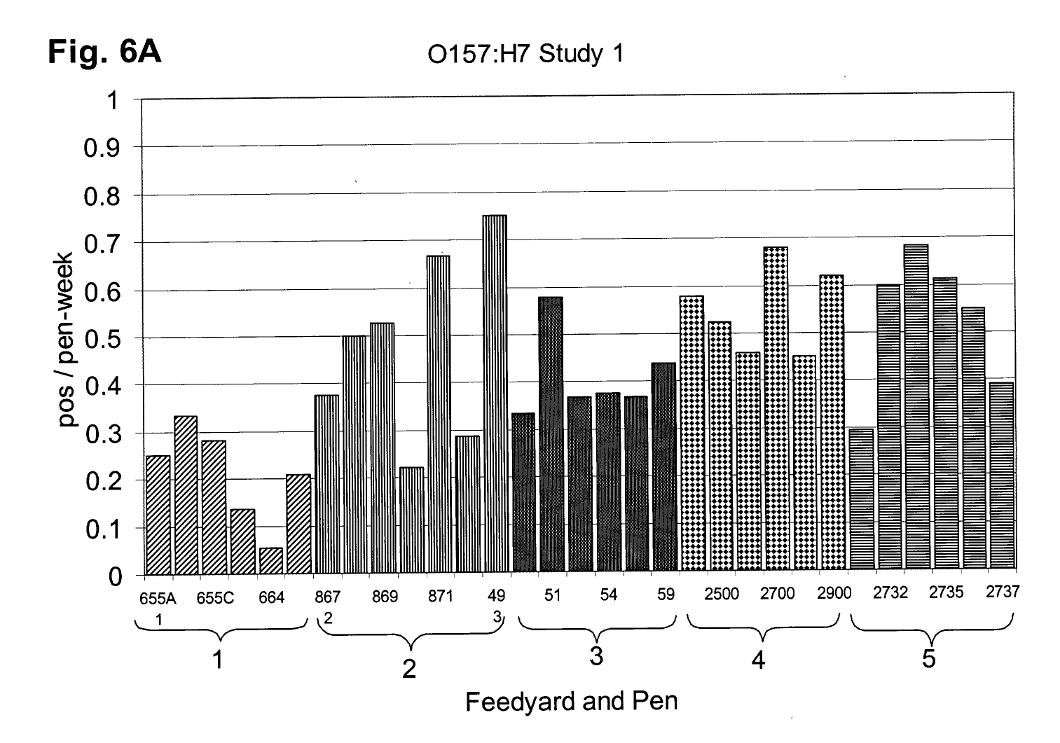

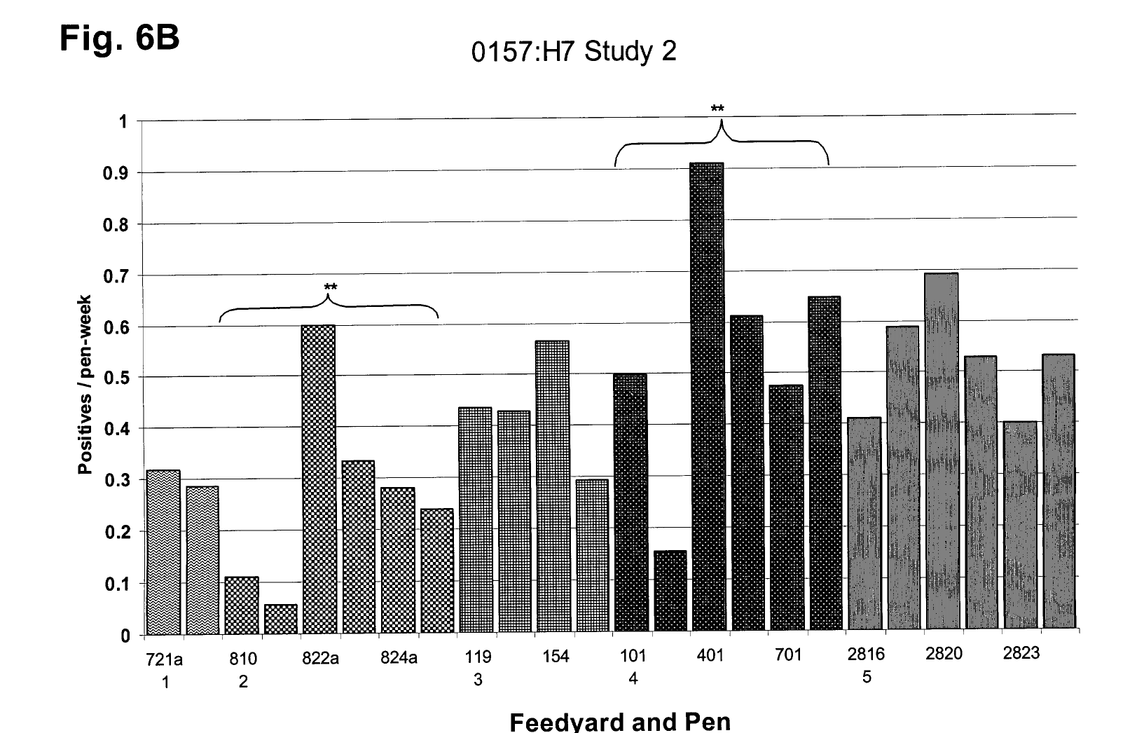

The results for E. coli 0157:H7 detection are shown in FIGS. 6A and 6B that depict the number of positives per pen-week for Study 1 and Study 2, respectively. As can be seen form the Figures, there were significant differences among feedyards in both studies (p<0.0001). However, while there were no differences among pens within feedyards in Study 1, pens within two feedyards in Study 2 differed in positives/pen-week (p<0.01)(marked with asterisks). It should be noted that in both studies, E. coli 0157:H7 was recovered at least once from each pen.

In assaying for the presence of Salmonella spp., 142 (23%) pen-weeks tested positive in Study 1, whereas 78 (18%) pen-weeks tested positive in Study 2. There was no significant difference between the percentages of Salmonella positive pen-weeks of the two studies (p=0.10).

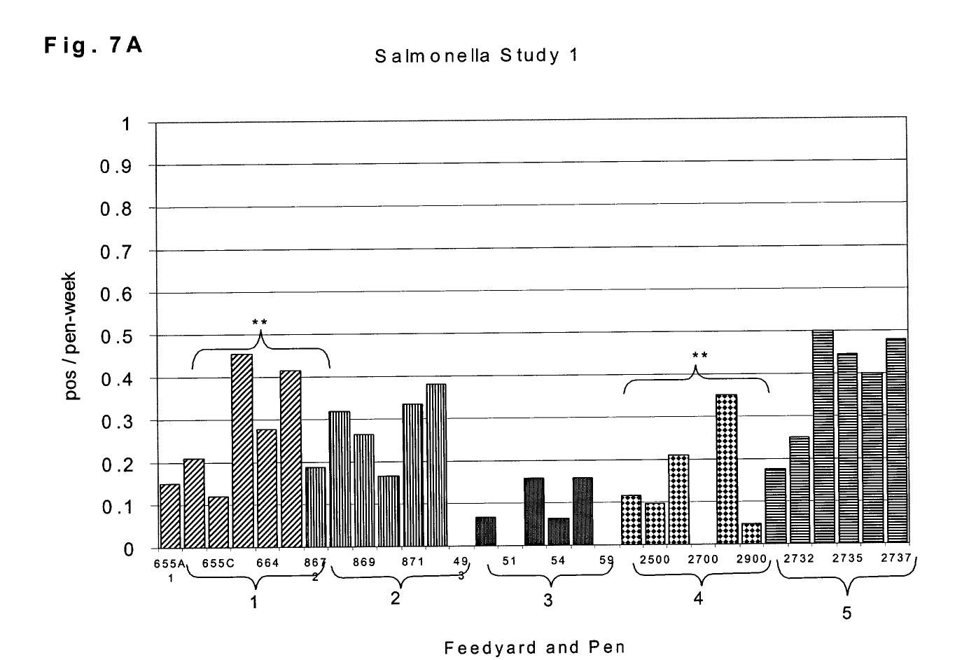

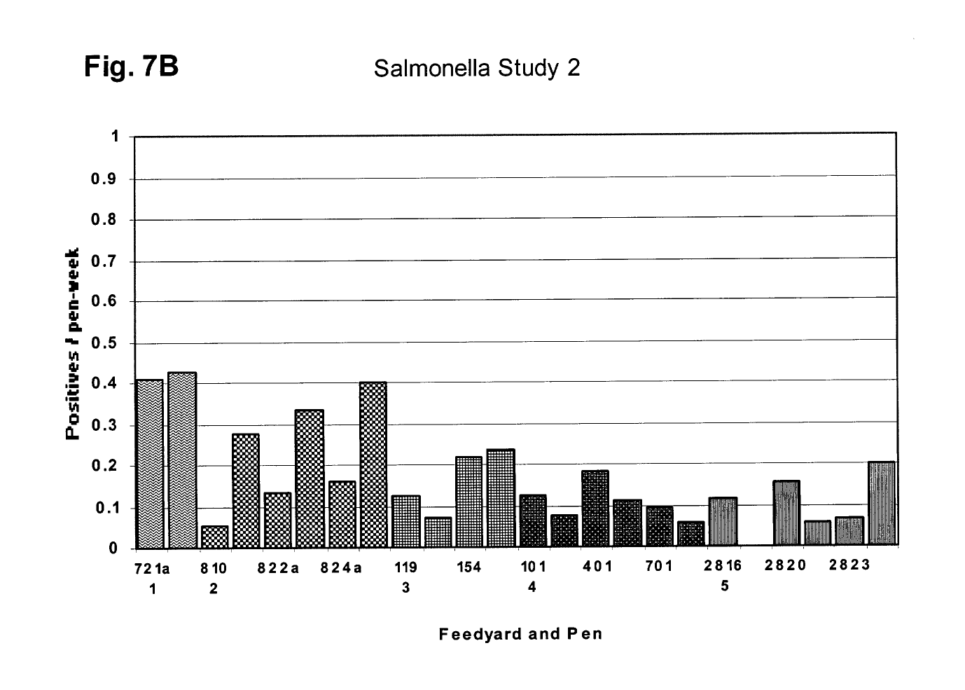

The results for Salmonella spp. are depicted in FIGS. 7A and 7B. Salmonella was recovered at least once from 27 pens in Study 1 (87%) and from 23 pens in Study 2 (96%). As seen with E. coli sampling, there were significant differences among feedyards in both studies (p<0.0001). In addition, pens differed within 2 feedyards (p<0.05) in Study 1 (marked with asterisks), whereas there were no differences among pens within feedyards in Study 2.

In light of the detailed description of the invention and the examples presented above, it can be appreciated that the several aspects of the invention are achieved.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventor does not intend to be bound by those conclusions and functions, but puts them forth only as possible explanations.

It is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the spirit and scope of the following claims.

What is claimed is:

1. A method to detect the presence of a microorganism in an animal, the method comprising:
(a) placing a device at a location within an area in which the animal resides to induce the animal to initiate contact with the device; and
(b) determining the presence of the microorganism on the device.

2. A method to detect the presence of an agent in an animal, the method comprising:
(a) placing a device at a location within an area in which the animal resides to induce the animal to initiate contact with the device; and
(b) determining the presence of the agent on the device.

3. The method of claim 1 wherein the microorganism is selected from the group consisting of bacterium, virus, protozoa and fungi.

4. The method of claim 3 wherein the microorganism is a bacterium.

5. The method of claim 4 wherein the bacterium is a human food-borne pathogen.

6. The method of claim 5 wherein the human food-borne pathogen is selected from the group consisting of Campylobacter jejuni, Salmonella, Escherichia coli, Listeria monocytogenes, and Vibrio.

7. The method of claim 6 wherein the human food-borne pathogen is Escherichia coli 0157:H7.

8. The method of claim 6 wherein the human food-borne pathogen is Salmonella.

9. The method of claim 2 wherein the agent is a chemical substance.

10. The method of claim 9 wherein the chemical substance is selected from the group consisting of arsenic, organophosphate, organochlorine, carbamate, antibiotic, or solvent.

11. The method of claim 1 or 2 wherein the animal resides with a group of animals.

12. The method of claim 1 or 2 wherein the device is selected from the group consisting of a rope, a ball, a sponge and artificial turf.

13. The method of claim 12 wherein the device is a rope.

14. The method of claim 13 wherein the rope is made of natural fiber, nylon, or cotton.

15. The method of claim 14 wherein the rope is made of natural fiber.

16. The method claim 1 or 2 wherein the animal contacts the device by rubbing any part of its body against the device or by placing the device in its mouth.

17. The method claim 1 or 2 wherein the animal is selected from the group consisting of cattle, sheep, swine, horse, goat, bison, deer, companion animal, and zoo animal.

18. The method of claim 17 wherein the animal is a cow.

19. The method of claim 1 or 2 wherein the area the animal resides is a feedlot or a pasture.

20. The method of claim 1 further comprising isolating the microorganism.

21. The method of claim 2 further comprising isolating the agent.

22. The method of claim 1 further comprising identifying the microorganism.

23. The method of claim 22 wherein the microorganism is identified by the polymerase chain reaction, an agglutination test, or by DNA sequencing.

24. The method of claim 2 further comprising identifying the agent.

25. The method of claim 24 wherein the agent is identified by chromatography or mass spectrometry.

26. The method of claim 1 wherein the microorganism is detected as a means to monitor the health status of the animal.

27. The method of claim 26 wherein the animal is a cow and the method is used to detect the presence of a microorganism selected from the group consisting of foot and mouth disease virus, bovine diarrhea virus, pseudorabies virus, and Salmonella spp.

28. The method of claim 2 wherein the agent is a mycotoxin.

29. The method of claim 28 wherein the mycotoxin is selected from the group consisting of aflatoxin, vomitoxin, and zearalone.
